(19) United States
(12) Patent Application Publication
(10) Pub. No.: US 2023/0173454 A1
(43) Pub. Date: Jun. 8, 2023

(54) PURIFICATION OF AROMATIC LIQUIDS

(71) Applicant: ARKEMA FRANCE, Colombes (FR)

(72) Inventors: Jérôme BLANC, PIERRE-BENITE Cedex (FR); Bernard MONGUILLON, COLOMBES CEDEX (FR); Ludivine BOUVIER, LACQ (FR); Ugo RAVON, LACQ (FR)

(21) Appl. No.: 17/998,934

(22) PCT Filed: Jul. 8, 2021
(86) PCT No.: PCT/FR2021/051267

§ 371 Date: Nov. 16, 2022

(30) Foreign Application Priority Data
Jul. 10, 2020 (FR) FR2007312

(51) Int. Cl. B01J 20/16; C07C 7/13

(52) U.S. Cl. CPC B01J 20/16; C07C 7/13

(57) ABSTRACT

The present invention concerns a process for purifying an aromatic liquid compound, said process comprising at least a step in which said aromatic liquid compound is contacted with a zeolitic adsorbent material.

The present invention also concerns the use of a zeolitic adsorbent material for purifying an aromatic liquid compound.

The present invention concerns the field of the purification of liquid compounds, more particularly of aromatic liquid compounds, and more particularly still of liquid compounds comprising at least one, preferably at least two, aromatic ring(s).

There are many fields of application currently using aromatic liquid compounds. The aromatic liquid compounds are in some cases subject to various stresses, and more particularly to greater or lesser thermal stresses, for longer or shorter durations, and often with repeated frequencies.

The aromatic liquid compounds, when subjected to thermal stresses, and more particularly to substantial and repeated thermal stresses, may tend to suffer degradation, so drastically reducing the useful life of said aromatic liquid compounds, while generating degradation products which may at best reduce the yield, and may even shorten the lifetime, of the aromatic liquid compound in the envisioned application and, in a more troublesome or even dangerous aspect, may lead to degradation products which are toxic for the environment, or even for living beings.

In particular, the degradations of the aromatic liquid compounds are generally observed over time at temperatures more or less close to their stability limit. The degradation products are usually classed into two categories: degradation byproducts with low boiling point and low flash point, termed “light” products, on the one hand, and the other, “heavy” degradation byproducts, which generally comprise one or more rings, optionally completely or partly unsaturated, which may be qualified hereinafter as “polyaromatic” products and “polycyclic” products.

The degradation byproducts with low boiling point and low flash point may give rise to problems of stability and/or safety during use, especially fire risks, pump cavitation problems, or else instances of pressure increase within apparatus.

These degradation byproducts with low boiling point and low flash point can usually be removed by drawing off the vapor phase present in said aromatic liquid compounds, especially when they are brought to temperatures higher than the boiling temperatures of the degradation products formed.

These “light” degradation products may also be separated by various means based on the differences in physicochemical properties with the aromatic liquid compounds of interest, and especially by settling, crystallization, recrystallization, etc., and combinations of two or more of these methods. Means of these kinds, though, remain highly time- and energy-consuming, so making them incompatible with profitable industrial applications.

The other, “heavy” polyaromatic and polycyclic degradation products may similarly be separated by various means based on the differences in physicochemical properties with the aromatic liquid compounds of interest, and for example by crystallization, recrystallization, etc., and combinations of two or more of these methods. As indicated above, though, means of these kinds remain highly time- and energy-consuming, so making them likewise incompatible with profitable industrial applications.

One means of overcoming these problems is generally to replace the spent aromatic liquid compound, i.e., the compound contaminated with the degradation byproducts. This solution generally involves the halting of the plants, the draining of the aromatic liquid compound comprising the impurities generated, and the treatment of said aromatic liquid compound contaminated with the impurities. As is readily appreciated, a solution of this kind represents a loss of time and of yield and hence an extra operating cost which may prove substantial.

Very often, producers treat the “light” byproducts by withdrawal of the vapor phase, as indicated above, whereas the “heavy” byproducts accumulate at a greater or lesser rate and very negatively impact the yields and/or the performances of the systems in which the aromatic liquid compounds are used.

Accordingly there remains a substantial need for solutions able to limit or retard the formation and/or accumulation of impurities, particularly the “heavy” byproducts generated during degradation of organic liquid compounds, so as to extend the lifetime of said aromatic liquid compounds in their uses, and so to avoid the discharge into the environment of toxic compounds, etc., and especially in uses where said aromatic liquid compounds are subject to more or less substantial, and repeated, thermal stresses.

It has surprisingly now been discovered that the aforementioned objectives can be resolved, entirely or at least in part, by virtue of the present invention. Still further objectives may become apparent in the description of the present invention that follows.

The inventors, then, have now discovered that the lifetime of aromatic liquid compounds can be greatly improved by trapping the degradation products formed in the course of the use of said aromatic liquid compounds, the trapping being performed by selective adsorption of said degradation products.

In a first aspect, therefore, the invention relates to a process for purifying an aromatic liquid compound, said process comprising at least a step in which said aromatic liquid compound is contacted with a zeolitic adsorbent material.

In the sense of the present invention, an “aromatic liquid compound” is a compound comprising at least one aromatic ring and preferably at least two aromatic rings, for example 2, 3 or 4 aromatic rings, and the partially or completely hydrogenated homologs thereof. By partially or completely hydrogenated homologs are meant that one aromatic ring (or two or more aromatic rings) is (or are) partially or completely hydrogenated. The aromatic liquid compounds of the present invention are defined, unless otherwise indicated, in their completely dehydrogenated forms, this signifying that the definition also embraces said organic liquid compounds in their completely or partially hydrogenated forms. Among these completely or partially hydrogenated forms, preference is given to the aromatic liquid compounds in which at least one aromatic ring is in its completely dehydrogenated form.

Entirely surprisingly, then, it has been discovered that the treatment of an aromatic liquid compound, optionally at least partially or completely hydrogenated, may be purified, and more particularly the amount of degradation products may be reduced substantially, or even completely, by contacting said liquid compound with a zeolitic adsorbent material -alternatively expressed, a material comprising at least one adsorbent exhibiting one or more zeolites, in any form, more particularly in the form of crystals and/or of zeolitic agglomerates.

According to one embodiment of the invention, the degradation products in the aromatic liquid compounds that can be removed, or at least whose amount can be greatly reduced, by virtue of the process of the present invention are generally and usually the most commonly encountered degradation products, including, as nonlimiting examples, benzene, toluene, dimethylbenzene, ethyltoluene, aniline, phenol, naphthalene, and their completely or partially hydrogenated forms, such as cyclohexane, methylcyclohexane, etc., and more generally still the aromatic aprotic apolar degradation products, or in completely or partially hydrogenated forms, of said aromatic liquid compounds.

The amount of the degradation products in the aromatic liquid compounds that can be removed, or at least whose amount can be greatly reduced, may vary within large proportions and is generally between 1 ppm and 10 000 ppm (by mass).

The aromatic liquid compound employed in the purification process of the present invention may be any type of compound that is liquid at ambient temperature and pressure (25° C., 1 atmosphere) and comprises at least one aromatic ring, in the nonhydrogenated form thereof, and preferably at least two aromatic rings, in the nonhydrogenated form thereof. The aromatic liquid compound useful within the process of the present invention may optionally be in at least partially, or even completely, hydrogenated form. These aromatic liquid compounds optionally in at least partially, or even completely, hydrogenated form are generally obtained from petroleum products and/or from products synthesized from petroleum products, but may also be obtained from renewable products and/or from products synthesized from renewable products.

It should be understood that the aromatic liquid compound employed in the process of the invention may take the form of a mixture of one or more aromatic liquid compounds, optionally partially or even completely hydrogenated, and for example the mixtures of aromatic liquid compounds obtained from petroleum products and/or from renewable products.

By aromatic liquid compounds obtained from petroleum products are meant, in the sense of the present invention, the products obtained from the separation and/or purification of petroleum, but also the compounds obtained from the synthesis of compounds bearing aromatic ring(s) of petroleum origin. By aromatic liquid compounds obtained from renewable products are meant, in the sense of the present invention, the products obtained from biomass, and in particular obtained from the extraction of wood (for example, lignin) and resinous products, and also the compounds obtained from syntheses of renewable products.

According to one preferred embodiment, the aromatic liquid compound which can be used in the process of the present invention conforms to the general formula (1):

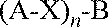

in which:
A and B, which are identical or different, represent, independently of one another, an aromatic ring, optionally completely or partially hydrogenated, optionally comprising at least, and preferably, one heteroatom, and optionally substituted by one or more saturated or partially or completely unsaturated hydrocarbon radicals comprising from 1 to 20 carbon atoms, preferably from 1 to 18 carbon atoms, more preferably from 1 to 12 carbon atoms, better still from 1 to 10 carbon atoms, even better still from 1 to 6 carbon atoms, typically from 1 to 3 carbon atoms,
X represents a spacer group, selected from a single bond, an oxygen atom, a sulfur atom, the divalent radical (CRR′)ₘ-, the divalent radical >C=CRR′, and the divalent radical -NR″-, or else
when n is other than 0 (zero), X forms, with the aromatic rings to which it is attached, a saturated or unsaturated ring comprising from 4 to 10 ring members, among which one or more of them may be a heteroatom selected from oxygen, nitrogen, sulfur, it being possible for said saturated or unsaturated ring to further be substituted by one or more hydrocarbon chains comprising from 1 to 30 carbon atoms, preferably from 1 to 10 carbon atoms,
R and R′, which are identical or different, are selected, independently of one another, from hydrogen and a saturated or partially or completely unsaturated hydrocarbon radical comprising from 1 to 6 carbon atoms, preferably from 1 to 3 carbon atoms,
R″ represents a saturated or partially or completely unsaturated hydrocarbon radical comprising from 1 to 6 carbon atoms, preferably from 1 to 3 carbon atoms,
m represents an integer of between 1 and 4, endpoints included, and
n can be equal to 0 or represents an integer equal to 1, 2 or 3, preferably equal to 1 or 2, with the restriction that, when n is equal to 0, B is substituted by one or more hydrocarbon radicals, as defined above.

The term “aromatic ring” is understood to mean monocyclic aromatic hydrocarbons and polycyclic aromatic hydrocarbons, comprising from 6 to 20 carbon atoms, among which one or more of them may be heteroatoms chosen from oxygen, sulfur and nitrogen, preferably from sulfur and nitrogen, and more preferably nitrogen. A “polycyclic compound” is understood to mean the rings defined above, which are fused or condensed, for example two, or more preferably two or three or four, more preferably two or three, for example two, fused or condensed rings.

When n is equal to 0, the aromatic liquid compound of formula (1) defined above forms part of the class of the alkylbenzenes, which are optionally partially or completely hydrogenated. When n is equal to 2 or 3, the groups (A-X) may be identical or different.

In one preferred embodiment of the present invention, in the aromatic liquid compound of general formula (1), n is equal to 0 and the organic liquid of formula (1) is generally selected from linear alkylbenzenes, which are optionally completely or partially hydrogenated, and branched alkylbenzenes, which are optionally completely or partially hydrogenated, such as, for example and without limitation, alkylbenzenes, and homologs which are completely or partially hydrogenated, in which the alkyl part comprises from 10 to 20 carbon atoms. [0031] Such alkylbenzenes include, again without limitation, decylbenzene, dodecylbenzene, octadecylbenzene, and the completely or partially hydrogenated homologs thereof, to mention only a few of them.

In another preferred embodiment of the present invention, the aromatic liquid compound of general formula (1) exhibits at least two aromatic rings, and in that case n is other than 0 and B is substituted by a hydrocarbon radical. Preferably again, said hydrocarbon radical is an alkyl radical comprising from 1 to 6 carbon atoms, preferably from 1 to 4 carbon atoms, and preferably the alkyl radical is the methyl radical.

As indicated earlier, the aromatic liquid compound conforming to the general formula (1) above can be used alone or as mixtures of two or more thereof in any proportions. According to one preferred embodiment of the invention, the aromatic liquid compound employed in the process of the present invention may contain one compound bearing at least one aromatic radical, which is optionally completely or partially hydrogenated, or a mixture of two or more compounds bearing at least one aromatic radical, which is optionally completely or partially hydrogenated. As indicated earlier, the aromatic liquid compound employed in the process of the invention is liquid at ambient temperature and ambient pressure.

According to yet another preferred embodiment of the present invention, the aromatic liquid compound is selected from benzyltoluene (BT), dibenzyltoluene (DBT), the partially or completely hydrogenated homologs thereof, and also mixtures thereof in any proportions.

In a very particularly preferred embodiment, the aromatic liquid compound is selected from the organic fluids sold by Arkema under the trade names of the Jarytherm® range.

Other aromatic liquid compounds, and partially or completely hydrogenated homologs, suitable for the requirements of the present invention are, for example, those sold by Eastman, especially under the trade name Marlotherm®.

Mention may be made, as yet other examples of aromatic liquid compounds suitable for the requirements of the present invention, of the following:
diphenylethane (DPE) and isomers thereof, more particularly 1,1-DPE (CAS 612-00-0), 1,2-DPE (CAS 103-29-7) and mixtures thereof (notably CAS 38888-98-1), such organic liquids being available commercially or described in the literature, for example in document EP 0 098 677,
ditolyl ether (DT) and isomers thereof, more particularly those corresponding to the numbers CAS 4731-34-4 and CAS 28299-41-4 and mixtures thereof, these notably being commercially available from Lanxess, under the name Diphyl DT,
phenylxylylethane (PXE) and its isomers, more particularly those corresponding to the numbers CAS 6196-95-8 and CAS 76090-67-0 and mixtures thereof, in particular commercially available from Changzhou Winschem, under the trade name PXE Oil,
monoxylylxylenes and dixylylxylenes, isomers thereof and mixtures thereof (CAS 186466-85-3),
1,2,3,4-tetrahydro-(1-phenylethyl)naphthalene (CAS 63674-30-6), this product being commercially available in particular from Dow under the reference Dowtherm™ RP,
diisopropylnaphthalene (CAS 38640-62-9), available in particular from Indus Chemie Ltd under the brand name KMC 113,
monoisopropylbiphenyl and isomers thereof (CAS 25640-78-2), notably available under the trade name Wemcol,
phenylethylphenylethane (PEPE) and its isomers (CAS 6196-94-7), in particular available from Changzhou Winschem or Yantai Jinzheng,
N-ethylcarbazole, notably available from Allessa GmbH,
phenylpyridines, tolylpyridines, diphenylpyridines, dipyridylbenzenes, dipyridinetoluenes,
and the partially or completely hydrogenated homologs thereof,
and mixtures of two or more of them, in any proportions,
to mention only the main organic liquids known and usable in the context of the present invention.

As indicated earlier, the process of the invention allows an organic liquid compound to be purified by contacting of said liquid compound with a zeolitic adsorbent material. The zeolitic adsorbent materials, these being materials comprising one or more zeolites, are well known to the skilled person for the removal of small molecules, generally present in the form of traces, from gaseous or liquid streams.

Moreover, zeolitic adsorbent materials usually comprise synthetic zeolites, which, by virtue of the wide variety of processes by which they are prepared, offer a great diversity of parameters that are amenable to fine adjustment, such as, for example, the thermal stability, the mechanical strength, or else the capacity for regeneration, in order to meet the specific criteria required for the envisaged use.

The zeolitic adsorbent materials which can be used in the context of the present invention may be of any types well known to the skilled person. The most suitable zeolitic adsorbent materials include natural or synthetic zeolites, and more particularly the zeolitic adsorbent materials selected from natural zeolites, as for example chabazite, and from zeolites of type LTA, zeolites of type FAU, zeolites of type EMT, zeolites of type MFI, and zeolites of type *BEA. These various types of zeolites are readily available to those skilled in the art commercially or are readily synthesizable by means of known procedures available in the scientific literature and in the patent literature. Moreover, the different types of zeolite are clearly defined and set out, for example, in the “Atlas of Zeolite Framework Types”, 5th edition, (2001), Elsevier.

For the requirements of the present invention, it is possible, as zeolitic adsorbent materials, to use mixtures of two or more zeolites, in any proportions. It is also possible to use hierarchically porous homologs of the aforementioned zeolites (known as “HPZs”) which are generally obtained by direct synthesis, notably using sacrificial agents, as described, for example, in patent applications WO 2015/019013 or WO 2007/043731, or else by surface post-treatment, as described, for example, in WO 2013/106816.

The zeolites listed above may be used in their “native” form, i.e., in the form of crystals, but are preferably used in the form of zeolite crystal agglomerates with one or more binders, by techniques well known to the skilled person, and especially by agglomeration of zeolite crystals with an agglomeration binder. The agglomeration binder may be of any type enabling the agglomeration and cohesion of the zeolite crystals and is generally selected from mineral clays, of which nonlimiting examples include kaolin, kaolinite, attapulgite, sepiolite, clinoptilolite, etc., and also mixtures of two or more of these clays, in any proportions.

Advantageously, then, the zeolite crystals are agglomerated with at least one agglomeration binder, and, if needed or desired, one or more additives well known to the skilled person, before being dried and/or baked and/or calcined.

The additives are likewise well known to the skilled person and their nature and amount thereof added may vary within wide proportions according to the desired or required effect. Examples of additives which may be used with the agglomeration binders include, without limitation, the surface passivation additives whose function is to manage the surface reactivity of the agglomerates and/or enhance their separation selectivity, tetrasodium pyrophosphate (TSPP) for example, and rheological additives, granulation additives, etc., and also mixtures of two or more of them.

The agglomerated zeolite crystals may also be engaged in an operation of zeolitization, also well known to the skilled person, which involves converting some or all of the agglomeration binder into zeolitic crystalline material, so as to augment the adsorption capacities of said agglomerates. The techniques of zeolite crystal agglomeration, drying, baking, calcination, and zeolitization are comprehensively described in both the scientific and the patent literature, and for example in applications WO1999/010096 and WO2000/050166.

The zeolites (crystals and agglomerates) indicated above generally and usually comprise cations to provide them with electronic neutrality. The most commonly used cations are selected from - as nonlimiting examples - alkali metals, alkaline earth metals and transition metals, and more particularly from cations of sodium, potassium, calcium, barium, strontium, magnesium, iron, copper, and silver. The zeolitic adsorbent materials which can be used in the context of the present invention may of course contain one or more of the cations listed above.

The presence of cations in the zeolitic adsorbent materials which can be used in the context of the present invention results either directly from the synthesis of said adsorbent materials, particularly the sodium cation for zeolites prepared from sodium-containing solutions, or via one or more cation exchange operations, by conventional techniques well known to those skilled in the art, where said exchanges may be carried out on the initial zeolite crystals and/or on the zeolite crystal agglomerates, before and/or during and/or after the shaping thereof, preferably before and/or after the shaping thereof.

The zeolitic adsorbent material which can be used in the context of the present invention may, indeed, if necessary or desired, and usually, be shaped, by any technique known to those skilled in the art, and more particularly by extrusion, granulation, etc., for shaping to forms such as beads, extrudates, etc., such as, for example, monolithic solids and membranes.

According to one embodiment of the process of the present invention, preference is given to using zeolitic adsorbent materials comprising one or more zeolites selected from:
LTA zeolites, preferably 5A zeolites, more particularly those comprising calcium cations, and the homologs thereof with hierarchical porosity (homologous zeolites comprising mesopores and micropores),
zeolites of type FAU, more particularly zeolites LSX, MSX, X and Y, and more particularly zeolites having an atomic ratio Si/AI of between 1 and 3, and the homologs thereof with hierarchical porosity (homologous zeolites comprising mesopores and micropores), as for example described in applications WO2015/019013, WO2015/019014, WO2015/028740, and WO2015/028741,
zeolites of type FAU, and more particularly zeolites having an atomic ratio Si/AI of strictly more than 3, and for example USY zeolites and dealuminated Y zeolites,
EMT zeolites or EMT-FAU intergrowth zeolitic phases, having an atomic ratio Si/AI of between 1 and 4, and the hierarchically porous homologs thereof (homologous zeolites comprising mesopores and micropores), as for example described in application WO2014/177567A1,
zeolites of type MFI, typically zeolites having an atomic ratio Si/AI of between 8 and 500, preferably between 8 and 250, more preferably between 8 and 100, advantageously between 8 and 50, better still between 8 and 40, and especially ZSM-5 zeolites, and the hierarchically porous homologs thereof (homologous zeolites comprising mesopores and micropores), and
zeolites of type *BEA, typically BETA zeolites having an atomic ratio Si/AI of more than 7, and preferably an atomic ratio Si/AI of between 8 and 20.

In one preferred embodiment, a zeolitic adsorbent material especially suitable for the needs of the process according to the present invention is a material comprising a zeolite of type FAU, comprising one or more cations selected from Na, K, Ba, Ca, Mg, Li, Sr, Ag, Cu, and more particularly NaX, BaX, BaKX, NaCaX, CaBaNaX, NaY, BaY, NaKY, BaKY, and mixtures thereof. These zeolites are available commercially and most of them are sold by Arkema.

The process for purifying an aromatic liquid compound according to the present invention thus comprises at least one step in which said liquid compound is contacted with a zeolitic adsorbent material as has just been defined. It should be understood that the process of the present invention employs one or more zeolitic adsorbent materials as have just been defined.

This contacting step may advantageously be carried out at a temperature of between -20° C. and 250° C., preferably between -15° C. and 150° C., preferably between -10° C. and 100° C., preferably between -5° C. and 80° C., preferably between -5° C. and 50° C., advantageously at ambient temperature, in other words at the working temperature, and more specifically without supply of heat or cold, for obvious reasons of the economic efficiency of the process of the invention.

Similarly, the contacting step may be carried out under pressure, at atmospheric pressure, or under reduced pressure, or even under vacuum. Operation preferably takes place, however, at atmospheric pressure, or under a pressure of possibly up to 20 bar (2 MPa), preferably 2 bar (200 kPa), and especially preferably under atmospheric pressure, i.e., at the working pressure, and more specifically without supply of pressure or reduced pressure, except for the differences in pressure resulting from devices such as pumps, valves, etc., for obvious reasons of the economic efficiency of the process of the invention.

The contacting time may vary within wide proportions, in particular depending on the nature and the amount of the impurities to be removed, on the nature and the amount of the zeolitic adsorbent material used, on the nature and the amount of the liquid to be purified, and on the type of contacting system used. In addition, the contacting time varies as a function of the temperature and pressure which are applied.

The contacting with the zeolitic adsorbent material may be carried out according to any method well known to those skilled in the art, continuously or in batch mode, and for example by passage, either forced (pumps) or by gravity, of the liquid through said zeolitic adsorbent material, such as in a packed column, or else by simple contact in a reactor, such as a reactor optionally equipped with a stirring system, etc.

More specifically, the step in the process of contacting the aromatic liquid to be purified with at least one zeolitic adsorbent material may be carried out in various static (or batch), dynamic, semicontinuous or continuous processes. For the latter processes, the stream to be purified generally passes through a bed of adsorbent on which the pollutants are retained selectively according to specific criteria such as, for example, the type of pollutant (polarity, diameter, steric bulk), the type of stream (gas, liquid), and operating conditions (temperature, pressure), etc.

The contacting step may thus take place singly or multiply, in batch mode and/or statically, in storage vats, with or without stirring, dynamically or continuously. This purification step preferably takes place before any step of storage of the liquid to be treated, and preferably dynamically through a bed of adsorbent, preferably dynamically through a fixed bed of adsorbent. Accordingly, and as nonlimiting examples, the contacting step in the process of the invention may be performed in batch mode, and in that case one embodiment comprises depositing a bed of adsorbent at the base of a container in which the aromatic liquid to be purified is stored, for a time which is variable according to the degree of pollution and the type of pollutants to be removed. This time, indeed, may vary in wide proportions, and is generally between a few minutes and a few days - for example, between 1 hour and 48 hours.

Alternatively, the contacting step may be performed continuously, by any known dynamic process for which the liquid to be purified passes through a bed of zeolitic adsorbent material, under the temperature and pressure conditions indicated earlier. The flow rate of continuous passage of said liquid through said bed of adsorbent may vary in wide proportions depending on the degree of pollution and the type of pollutants to be removed, but is generally adjusted to permit a contact time of generally between a few minutes and a few days - for example, between 1 hour and 48 hours. The bed of zeolitic adsorbent material may be of any type well known to those skilled in the art, and more particularly a fixed bed, fluidized bed or moving bed (simulated or otherwise). In the case of continuous contacting, preference is given to employing a fixed bed with regeneration of the screen or operation in two adsorbers, a first working in adsorption and a second working in desorption/regeneration.

According to one especially advantageous embodiment of the invention, indeed, the zeolitic adsorbent material may be desorbed and/or regenerated, in batch mode or continuously, by conventional desorption and regeneration techniques, and especially by heat treatment and/or by means of one or more desorption solvents.

Accordingly the process of the present invention employs at least one zeolitic adsorbent material as indicated above which may be present in various forms, and especially a bed of adsorbent, for example one or more types of zeolite in the form of a mixture of crystals or agglomerates, or else a plurality of beds of identical or different adsorbents in the same adsorber, where one or more adsorbers may be used, in series and/or in parallel, so as to maximize the selectivity and extent of removal of the impurities present in the aromatic fluids, and especially the monocyclic impurities, such as, for example, toluene, benzene, methylcyclohexane, dimethylbenzene, ethyltoluene, aniline, phenol, naphthalene, and the at least partially or completely hydrogenated homologs thereof.

More specifically still, the process of the invention comprises at least the following steps:
a) providing an aromatic liquid comprising at least one impurity,
b) contacting said aromatic liquid with at least one zeolitic adsorbent material,
c) recovering said aromatic liquid comprising said at least one impurity at a concentration by weight of less than 50%, preferably less than 40%, preferably less than 30%, more preferably less than 20% by weight relative to the level of impurity present in the liquid from step a), and
d) optionally regenerating and/or desorbing said at least one zeolitic adsorbent material.

The process of the present invention is especially suited to the purification of aromatic liquids comprising at least one aromatic ring, and preferably at least two aromatic rings, and polluted with one or more above-defined impurities such as byproducts generated during the breakdown of organic liquid compounds, and especially the monocyclic impurities, such as, for example, toluene, benzene, methylcyclohexane, dimethylbenzene, ethyltoluene, aniline, phenol, naphthalene, and the at least partially or completely hydrogenated homologs thereof, to cite only the principal representatives thereof, without limitation.

The process according to the present invention may thus be employed in a large number of fields of application, and especially the fields of application in which an aromatic liquid is subject to degradation conditions, such as, for example, thermal variations, whether substantial or otherwise, cyclical or otherwise, and chemical modifications, whether reversible or otherwise, etc.

Possible nonlimiting examples of such fields of application include fields in which aromatic liquids are used as heat transfer fluids, dielectric fluids, or else fields in which aromatic liquids are used as liquid organic hydrogen carriers (also referred to by the acronym “LOHC”), as for example described in application WO2014/082801.

The process of the invention, indeed, is especially suited to the purification of heat transfer liquids or LOHC liquids, and particularly to the aromatic liquids benzyltoluene and dibenzyltoluene, alone or mixed in any proportions. According to one particularly preferred embodiment, the process of the invention relates to the purification of benzyltoluene or dibenzyltoluene, or of mixtures thereof, by contacting with one or more zeolitic adsorbents based on one or more zeolites of type FAU, as indicated above.

As indicated above, the process of the invention may be implemented in batch mode or continuously, one or more times, according to the requirements encountered in the relevant field of application.

Accordingly, and for example in the case of use as an LOHC, the organic liquid may be purified one or more times, before or after one or more of the steps in the process, and for example before a dehydrogenation step and/or before a hydrogenation step.

Lastly, in another aspect, the present invention relates to the use of a zeolitic adsorbent material as has just been defined for purifying an aromatic liquid compound, as defined earlier on above.

1–11. (canceled)

12. A process for purifying an aromatic liquid compound, said process comprising at least a step in which said aromatic liquid compound is contacted with a zeolitic adsorbent material.

13. The process as claimed in claim 12, wherein said aromatic liquid comprises at least one aromatic ring, in the nonhydrogenated form thereof.

14. The process as claimed in claim 12, wherein the aromatic liquid compound used in the process corresponds to the general formula (1): 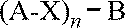
in which:
A and B, which are identical or different, represent, independently of one another, an aromatic ring, optionally completely or partially hydrogenated, optionally comprising at least, and optionally substituted by one or more saturated or partially or completely unsaturated hydrocarbon radicals comprising from 1 to 20 carbon atoms,
X represents a spacer group, selected from a single bond, an oxygen atom, a sulfur atom, the divalent radical (CRR′)ₘ-, the divalent radical >C=CRR′, and the divalent radical -NR″-, or else
when n is other than 0 (zero), X forms, with the aromatic rings to which it is attached, a saturated or unsaturated ring comprising from 4 to 10 ring members, among which one or more of them may be a heteroatom selected from oxygen, nitrogen, sulfur, it being possible for said saturated or unsaturated ring to further be substituted by one or more hydrocarbon chains comprising from 1 to 30 carbon atoms,
R and R′, which are identical or different, are selected, independently of one another, from hydrogen and a saturated or partially or completely unsaturated hydrocarbon radical comprising from 1 to 6 carbon atoms,
R″ represents a saturated or partially or completely unsaturated hydrocarbon radical comprising from 1 to 6 carbon atoms,
m represents an integer of between 1 and 4, endpoints included, and
n can be equal to 0 or represents an integer equal to 1, 2 or 3, with the restriction that, when n is equal to 0, B is substituted by one or more hydrocarbon radicals, as defined above.

15. The process as claimed in claim 12, wherein the aromatic liquid compound is selected from the group consisting of:
benzyltoluene (BT), dibenzyltoluene (DBT), the partially or completely hydrogenated homologs thereof, and also mixtures thereof in any proportions,
diphenylethane (DPE) and isomers thereof, more particularly 1,1-DPE, 1,2-DPE and mixtures thereof,
ditolyl ether (DT), isomers thereof, and mixtures thereof,
phenylxylylethane (PXE), isomers thereof, and mixtures thereof,
monoxylylxylenes and dixylylxylenes, isomers thereof and mixtures thereof,
1,2,3,4-tetrahydro(1-phenylethyl)naphthalene,
diisopropylnaphthalene,
monoisopropylbiphenyl and isomers thereof,
phenylethylphenylethane (PEPE) and isomers thereof,
N-ethylcarbazole,
phenylpyridines, tolylpyridines, diphenylpyridines, dipyridylbenzenes, dipyridinetoluenes,
and the partially or completely hydrogenated homologs thereof,
and mixtures of two or more of them, in any proportions.

16. The process as claimed in claim 12, wherein the zeolitic adsorbent material is a material comprising at least one adsorbent exhibiting one or more zeolites, in the form of crystals and/or of zeolitic agglomerates.

17. The process as claimed in claim 12, wherein said zeolitic adsorbent material is selected from natural or synthetic zeolites.

18. The process as claimed in claim 12, wherein said zeolitic adsorbent material comprises at least one cation selected from the group consisting of alkali metals, alkaline earth metals and transition metals.

19. The process as claimed in claim 12, wherein said zeolitic adsorbent material comprises one or more zeolites selected from the group consisting of:
LTA zeolites,
zeolites of type FAU, selected from the group consisting of zeolites LSX, MSX, X and Y, and having an atomic ratio Si/Al of between 1 and 3, and the homologs thereof with hierarchical porosity,
zeolites of type FAU, selected from zeolites having an atomic ratio Si/Al of strictly more than 3,
EMT zeolites or EMT-FAU intergrowth zeolitic phases, having an atomic ratio Si/Al of between 1 and 4, and the hierarchically porous homologs thereof,
zeolites of type MFI having an atomic ratio Si/Al of between 8 and 500, and the hierarchically porous homologs thereof, and
zeolites of type *BEA, having an atomic ratio Si/Al of more than 7.

20. The process as claimed in claim 12, wherein said zeolitic adsorbent material is a material comprising a zeolite of type FAU, comprising one or more cations selected from Na, K, Ba, Ca, Mg, Li, Sr, Ag, Cu, and mixtures thereof.

21. The process as claimed in claim 12, comprising at least the following steps:
a) providing an aromatic liquid comprising at least one impurity,
b) contacting said aromatic liquid with at least one zeolitic adsorbent material,
c) recovering said aromatic liquid comprising said at least one impurity at a concentration by weight of less than 50% by weight relative to the level of impurity present in the liquid from step a), and
d) optionally regenerating and/or desorbing said at least one zeolitic adsorbent material.

22. The process as claimed in claim 17, wherein said zeolitic adsorbent material is selected from the group consisting of chabazite, zeolites of type LTA, zeolites of type FAU, zeolites of type EMT, zeolites of type MFI, and zeolites of type *BEA.
